(19) United States
(12) Patent Application Publication
(10) Pub. No.: US 2019/0170719 A1
(43) Pub. Date: Jun. 6, 2019

(54) WINE PROBE

(71) Applicant: OENONEO, Sainte-Marie-d'Alloix (FR)

(72) Inventors: Bastien GUILLEBASTRE, Sainte-Marie-d'Alloix (FR); Fabien MUNOZ, Dagnieux (FR)

(73) Assignee: OENONEO, Sainte-Marie-d'Alloix (FR)

(21) Appl. No.: 16/314,655

(22) PCT Filed: Jun. 29, 2017
(86) PCT No.: PCT/FR2017/051763

§ 371 Date: Dec. 31, 2018

(30) Foreign Application Priority Data
Jun. 29, 2016 (FR) 1656078

(51) Int. Cl. G01N 33/14; G01N 21/25

(52) U.S. Cl. CPC G01N 33/146; G01N 21/255

(57) ABSTRACT

A wine probe includes a sealed body configured to be immersed in wine and defining a through cavity of the body. The probe includes at least one light source positioned on a first sidewall of the cavity configured to consecutively emit at least two radiations in at least two different wavelengths. The probe also includes at least one light sensor positioned on a second side wall opposite the first, facing the light source and configured to measure at least two light intensities from the radiations which have passed through the cavity. The probe also includes a control device configured to initiate emission of the radiations and to collect the measurements made by the sensor. The control device is configured to transmit data relative to the analysed wine to a readout from the measurements. Also, a method measures quantities relative to a wine by the probe.

### FIELD OF THE INVENTION

The invention relates to a wine probe and to the method for measuring quantities characteristic of the wine from one such probe.

### STATE OF THE ART

Depending on the terroir, the grape variety and the vintage, a wine can have very different characteristics (maturity, acidity, astringency, etc.). For example, Bordeaux wines and Burgundy wines are very different because the former generally correspond to an assembly of grape varieties, whereas the latter are more often than not mono-varietal wines different from those grown in the Bordeaux region. In addition, the same grape variety can produce different fruits depending on the terroir and the climatic conditions. Finally, wine growers do not manufacture and age the wines in the same way. This results in a large disparity of tastes and textures of the wines.

A first wine drinker may therefore have a preference for a certain category of wine, whereas a second wine drinker may have a preference for another category of wine.

Very often, wine drinkers use guidebooks edited by specialists, which give the characteristics of the wines by vineyard estate and by year. These guidebooks are very useful for choosing a wine, but the specialist's opinion may not be confirmed by the oenophile's palate when tasting.

After he has purchased it, the wine drinker has no other choice than to taste his wine, with the risk of its characteristics being very different from those initially expected.

It is known from the document “Demonstration of spectrophotometric rapid analysis of red wine” by Mikhail Proskurnin, 23 Nov. 2014 Third International Conference of CIS IHSS HIT-2014 that the spectral parameters of a red wine change according to the wavelength. The document indicates that the data relative to the 280 nm wavelength is linked to the phenolic groups, the data relative to the 420 nm wavelength is linked to the tannins and the data relative to the 280 nm wavelength is linked to the anthocyanes.

This teaching proposes a diffraction method by means of a spectrophotometer that comprises a halogen lamp, a slit, a monochromator, lenses and a set of mirrors in order to monitor the whole of the visible spectrum and beyond. This solution is cumbersome, energy consuming and costly.

The document presents an ATR probe presenting a cavity through which white light passes. Such a probe requires prior sample dilution work, which renders its use less advantageous.

It should also be underlined that the document presents two strictly identical graphs, on pages 22 and 23, so that no information can be drawn from these graphs.

### OBJECT OF THE INVENTION

One object of the invention consists in proposing a wine probe that makes it possible to quickly determine data relative to a wine, in non-destructive manner, without sampling, and without chemical interaction, and to transmit the latter to a user via a readout.

For this purpose, the wine probe comprises:
a sealed body configured to be immersed in wine and defining a through cavity of the body,
at least one light source configured to consecutively emit at least two radiations in at least two different wavelengths, the light source being positioned on a first side wall of the cavity of the body,
at least one light sensor positioned on a second side wall of the cavity opposite the first side wall, the light sensor being placed facing the light source, the light sensor being configured to measure at least two light intensities from the at least two radiations emitted by the light source which have passed through the cavity,
a control device configured to initiate emission of the at least two light radiations and to collect the light intensity measurements made by the sensor, the control device being configured to transmit data relative to the analysed wine to a readout from the at least two light intensity measurements.

According to a particular embodiment, the cavity can be L-shaped in a cutting plane orthogonal to the longitudinal axis of the body.

According to one feature of the invention, the cavity can have a width comprised between 1 and 10 mm, preferentially between 1 and 5 mm, and ideally equal to 2 mm.

The control device can advantageously be configured so that
the light source is configured to successively emit the at least two radiations at a frequency of more than 30 Hz,
the light sensor is configured to perform the at least two light intensity measurements at a frequency of more than 30 Hz,
the control device collects the at least two measurements at a frequency of more than 30 Hz.

The control device is, in this case, configured to collect several series of at least two light intensity measurements, and to calculate a statistical mean of each of the measured light intensities.

The light source of the probe can further comprise at least two different monochromatic sources.

When the light source is configured to consecutively emit at least three radiations in at least three different wavelengths, the control device can advantageously transform the different measured light intensities into coordinates in a Red, Green and Blue framework. As an alternative, it can transform the different measured light intensities into coordinates in a Hue/Saturation/Value and/or CIELAB colorimetric framework. It can also represent the measured light intensities in a framework representing the acidity, the body and the maturity of the analysed wine. These coordinates can be displayed on a readout.

The invention also relates to a method for measuring data relative to a wine comprising the following steps:
providing a wine probe comprising the above-mentioned features,
immersing the cavity of the probe in the wine so that the light emitted by the light source passes through the wine until it reaches the light sensor,
emitting at least two radiations in at least two different wavelengths and measuring the at least two light intensities received by the light sensor for the at least two radiations,
collecting the at least two measurements measured by the light sensor,
transmitting data relative to the analysed wine to a readout from the at least two light intensity measurements.

The method can comprise the following specificities:
the light source can be configured to consecutively emit the at least three radiations in at least three different wavelengths,
the light sensor can be configured to measure the at least three radiations,
the relative data displayed can be the acidity, the body and the maturity, this data being calculated from the at least three light intensity measurements.

### BRIEF DESCRIPTION OF THE DRAWINGS

Other advantages and features will become more clearly apparent from the following description of particular embodiments of the invention given for non-restrictive example purposes only and represented in the appended drawings, in which:

FIG. 1 represents a perspective view of the wine probe in schematic manner,

FIGS. 2 and 3 represent cross-sectional views of two alternative embodiments of the cavity of the wine probe,

FIGS. 4 and 5 represent a front and a side cross-sectional view of the inner part of the wine probe,

FIG. 6 schematically illustrates a side view of the probe after assembly of the inner part with an outer cover.

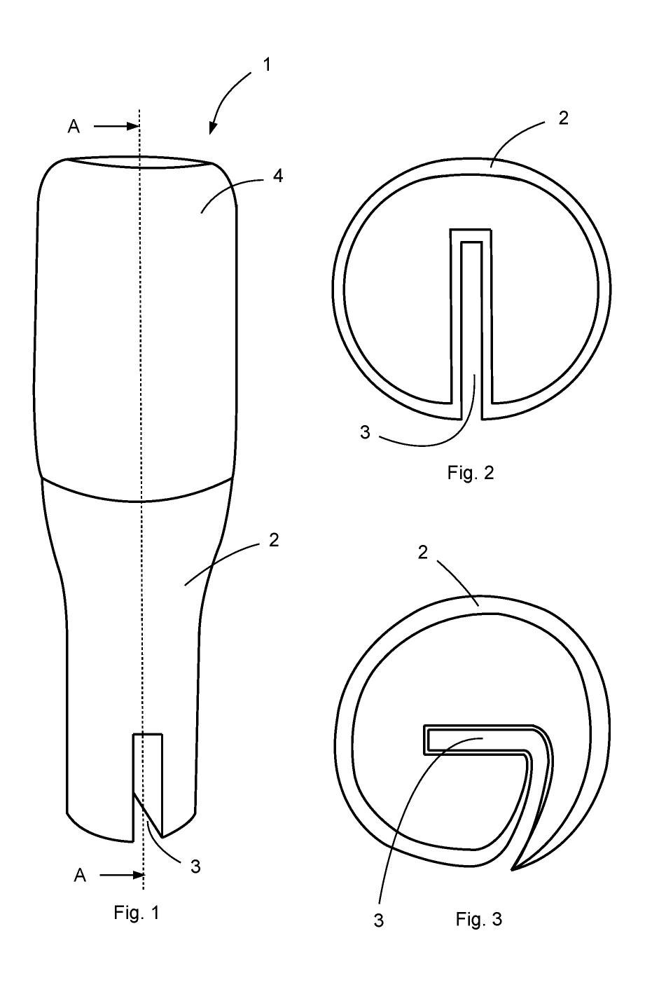

### DETAILED DESCRIPTION

The wine probe 1 is provided with a sealed body 2 that is able to be immersed in the wine without the latter penetrating into the probe 1, so that there is no chemical interaction between the probe 1 and the wine. The body 2 comprises a through cavity 3 inside which wine is contained when the measurements are performed. The walls of the cavity 3 can be translucent or transparent to allow light to pass, as will be seen further on.

The wine probe 1 advantageously comprises a cover 4 allowing access to components performing analysis of the characteristics of the wine and to a power supply means (not shown). The probe 1 can be supplied with electric power by an internal battery or by an external device such as a main power supply. According to a particular embodiment of the probe 1, the latter can be supplied with electric power by either of the two systems as the user desires.

The components performing analysis of the characteristics of the wine are fitted on a electronic card 5 and comprise at least one light source 6 able to consecutively emit on at least two different wavelengths. The light source or sources 6 are advantageously positioned against a first side wall of the cavity 3 so as to emit light in the direction of the cavity 3.

At least one light sensor 7 is positioned against a second side wall of the cavity opposite the first side wall. The light sensor 7 is advantageously positioned facing the light source 6 and is configured to measure the light intensity of the at least two radiations that have passed through the cavity 3.

According to an advantageous embodiment, there are as many light sensors 7 as there are light sources 6, and each light sensor 7 is placed facing a light source 6 so as to pick up the light emitted by the latter. According to the embodiment illustrated in the figures, the wine probe 1 comprises first and second light source 6 and light sensor 7 couples.

The electronic card 5 also comprises a control device 8 which is configured to turn the light source 6 on and to initiate emission of the at least two light radiations. It also retrieves the measurements made by the light sensor 7 for each of the at least two wavelengths emitted by the source, and transmits data relative to the analysed wine from the at least two light intensity measurements to a readout (not shown).

The readout can for example be a digital monitor positioned on the cover 4 or on the body 2 of the probe 1. The readout can also be an external device, for example a mobile phone or a computer. In this case, the probe is advantageously equipped with hardwired or wireless (wifi, bluetooth, NFC, etc.) transmission means 9.

According to a particular embodiment, the control device 8 can comprise an external means able to be directly controlled by a user. This external means can for example be a computer software or a smartphone application. In this case, the part of the control device 8 which is present on the electronic card 5 corresponds to components transmitting the emission order of the at least two light radiations by the light source 6, and retrieving the measurements made by the light sensor 7. The readout can then form an integral part of the computer software or of the smartphone application.

From a structural point of view, the width of the cavity 3 can advantageously be comprised between 1 and 10 mm. If the width of the cavity 3 was smaller than 1 mm, wine could remain in the cavity 3 by capillarity after the measurements have been made and bias future measurements made with the probe 1. If the width of the cavity 3 was larger than 10 mm, the light intensity reaching the light sensor 7 would be too weak for the wine to be able to be analysed in reliable manner. The width range comprised between 1 and 5 mm is a good trade-off as the measured light intensity is sufficient for the measurements to be precise. The cavity 3 preferentially has a width of 2 mm in order to obtain optimal measurements.

According to a particular embodiment (not represented in the figures) the cavity 3 can have a width that is not constant. In a plane orthogonal to the longitudinal axis AA of the probe 1, the cavity 3 can for example have a trapezoid shape the width of which is advantageously comprised between 1 and 10 mm. In this embodiment, it may be advantageous for the probe 1 to comprise first and second light source 6 and light sensor 7 couples. The latter can be positioned laterally against the side walls of the cavity 3 so that a first couple is separated by a first distance D1 and a second couple is separated by a second distance D2 different from the first distance, the two distances being comprised between 1 and 10 mm.

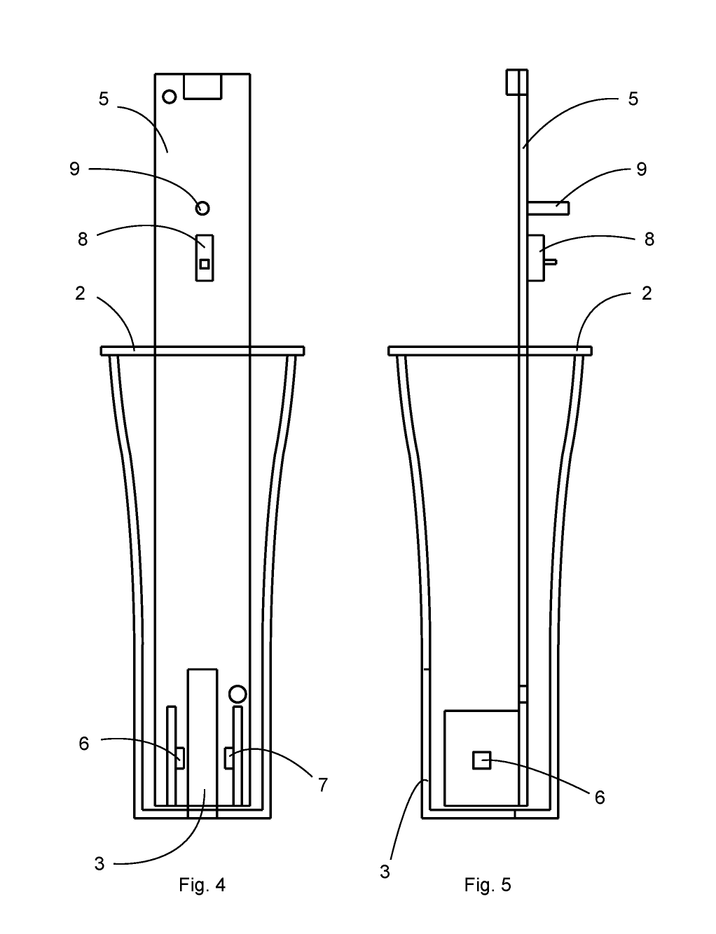

The cavity 3 can present different shapes illustrated in FIGS. 2 and 3. According to a first embodiment presented in FIG. 2, the cavity 3 can have a rectangular shape in a cutting plane orthogonal to the longitudinal axis AA of the wine probe 1. Cleaning of the cavity 3 is then made easier, thereby avoiding measurements being obtained that are inaccurate due to the residues of previous wine analyses.

According to another advantageous embodiment illustrated in FIG. 3, the cavity 3 can be L-shaped or in the form of a portion of a spiral in a cutting plane orthogonal to the longitudinal axis AA. This shape enables the effects of stray light sources such as daylight to be limited. In this way the measurements made by the light sensor 7 are more precise as the latter only receives the light emitted by the light source 6.

The light source 6 can advantageously emit at least two wavelengths, and advantageously wavelength triplets. Each wavelength can be emitted successively in order to perform consecutive measurements to analyse the wine, for example two consecutive measurements. The light source 6 can comprise as many monochromatic sources as emitted wavelengths, for example three monochromatic sources if three wavelengths are used. As an alternative, a single light source can emit light in several different wavelengths.

When the light source 6 is configured to emit three different wavelengths, the wavelengths can for example be 700 nm, 546.1 nm and 435.8 nm. These wavelengths correspond respectively to red, green and blue. These are the three prime colours which, when emitted simultaneously with an identical energy flux, give white.

The wavelength triplets can advantageously be emitted by the light source 6 at a frequency of more than 40 Hz. This frequency corresponds to the time period below which the human eye cannot tell the difference between two successive images, i.e. 25 ms. This phenomenon is also called persistence of vision or retinal persistence.

When the wavelength triplets emitted by the light source 6 correspond to red, green and blue, the user of the probe 1 has the impression that the light emitted by the source 6 is white as his eye cannot tell the difference between the wavelengths successively emitted for a given triplet.

When the wavelength triplets are emitted at a frequency of more than 30 Hz, the eye is under the impression that the wine probe 1 is emitting a glittering white light. This frequency is sufficient for the red, green and blue colours not to be apparent to the user's eye, the latter not being aware of how the probe 1 works. This also enhances visual comfort.

In response to a request from the control device 8, the light source 6 emits light in the direction of the cavity 3, the light passing through the wine that is to be analysed. The properties of the light emitted by the source 6 are modified by the presence of the wine, and the light sensor 7 measures the received light intensities. The measurements are then collected by the control device.

As the light source 6 emits at least two wavelengths, and preferentially wavelength triplets at a frequency of more than 30 Hz, the light sensor 7 is configured to perform the measurements at the same frequency. In less than 33 ms, it is therefore able to distinguish the received light intensities corresponding to the two or three wavelengths emitted by the source 6. The sensor 7 can preferentially provide triplets of measurements to the control device 8 at a frequency or more than 30 Hz, and the control device 8 is therefore configured to collect the measurements at a frequency or more than 30 Hz.

This configuration enables a large quantity of data to be collected in a minimum amount of time so as to enhance the precision of the data supplied to the user.

According to a particular embodiment, the control device 8 can be configured to calculate a statistical value from values measured for each of the three wavelengths emitted by the light source 6. The statistical value can be calculated from a predefined number of measurements made by the sensor 7, or at the end of a predefined time period. The statistical value can correspond to the mean values of the intensity measured for each wavelength. The number of measurements made to calculate the mean can be comprised between 25 and 50, and preferentially be equal to 30 so as to make quick and dependable measurements.

According to a specificity of the invention, the user can for example choose between different calculation methods to determine the statistical mean of the measured intensities. He/she can also choose the number of measurements made or choose the sampling time before the control device 8 calculates the mean triplet. The user can therefore advantageously tune the parameters of the probe 1 so that its use corresponds to his requirements.

The measurement frequency is such that, whatever the method used, the user can obtain the values characteristic of the wine almost instantaneously.

The measurements collected by the control device 8 can be represented in different frameworks. When the source 6 emits three monochromatic wavelengths able for example to be red, green or blue, the sensor measurements can be represented in the Red, Green, Blue (RGB) framework without requiring any specific mathematical processing. The data supplied to the user in this framework is however not really evocative of the properties of the wine.

For the user to be able to interpret the data supplied by the probe 1 more easily, the measurements encoded in the RGB framework can be expressed in another colorimetric framework, called Hue, Saturation, Lightness (HSL) and/or CIELAB. This framework can be visually represented by a double-cone having a white tip, the other tip being black, the sides of the cone corresponding to the visible wavelengths. In this framework, the hue corresponds to the colour in everyday language, i.e. the angular position on the double cone. The saturation corresponds to a distance with respect to an achromatic colour such as white, grey or black, i.e. the distance with respect to the longitudinal axis of the cone. And finally the lightness corresponds to the position of the colour between the black and white, i.e. the position along the longitudinal axis of the cone.

This colorimetric framework provides data that is more informative and more relevant for the user, but it does not correspond to the data conventionally provided to the wine drinker in guidebooks, such as the maturity, body or acidity.

Therefore, to remedy this problem, the control device 8 can comprise a computer designed to convert the collected measurements into a framework representing the maturity, body and acidity. These calculations can for example be based on Beer-Lambert's law or on a non-linear model. The maturity of the wine can for example be determined directly from the value of the hue in the HSL framework.

As an alternative, a learning method can be executed from analysis by means of the probe 1 of several wines the properties of which are known. The database constructed by this learning method can then be integrated in the control device 8. The latter then uses the collected measurements and the database to deduce the properties of an unknown wine therefrom.

Whatever the framework used to evaluate the measurements collected by the control device 8, the latter are displayed on the readout, which can be a digital monitor for example.

To use the probe 1, calibration has to be performed beforehand. This calibration can consist in measurement of the triplet of light intensities received by the sensor 7 when it receives the light from the source after the latter has passed through air, or water, for example mineral water or pure water. This calibration step enables the wavelengths emitted by the light source 6 to be determined with precision, hereby enabling any drift with respect to the scheduled wavelengths to be detected.

The calibration step of the probe 1 can be performed by the manufacturer or by the user in the course of the lifetime of the equipment. Regular calibration ensures the reliability of the measurements made.

When the wine probe 1 comprises a battery, the probe 1 can be configured to self-calibrate according to the quantity of electricity supplied by the battery.

To determine the data relative to the wine, the probe 1 first has to be immersed in the wine so that the wine flows into the cavity 3, and preferably fills the cavity up. In this way, the area of the cavity separating the light source 6 from the light sensor 7 is filled by the wine.

The user can then command turn-on of the light source 6 via the control device 8. The source emits at least two consecutive wavelengths so that the sensor 7 measures the received intensity in repetitive manner for each wavelength.

According to an alternative embodiment, the wine probe 1 can be in standby mode and switch automatically to active mode when it detects a fluid in the cavity 3. The measurements are then made and the probe 1 switches back to standby mode when the measurements have been completed.

The light emitted by the source 6 reaches the sensor 7 and its properties are modified by the presence of the wine. The measurements are collected by the control device and displayed via the readout.

Before to be displayed, the measurements can be averaged in order to provide the user with more reliable data. The statistical method used to calculate the mean values according to the wavelength can be chosen by the user by means of the control device 8.

When the light source 6 is configured to emit at least three different wavelengths, and when the light sensor 7 can measure at least three different light intensities, the displayed measurements can be encoded in different frameworks such as those mentioned above. The readout can for its part be configured to enable simultaneous display of the measurements in several frameworks. The displayed measurements can in particular be relative to the acidity, the body and the maturity of the analysed wine.

When the control device 8 is equipped with transmission means 9, the control device 8 can be configured to transmit the measured data over the internet in order to feed a common database identifying the properties of the wine for the benefit of web users.

A probe 1 is thus provided enabling rapid and non-destructive checking of the wine, performed without sampling or chemical interaction. The probe 1 can easily be used by beginners or by professional oenologists to check the quality of a wine and to acquaint themselves with its characteristics in objective manner before consuming it or selling it.

As illustrated in the different figures, the probe 1 is portable so that the whole of the probe is placed in the recipient containing the wine to be analysed. This solution is much more advantageous than solutions existing in the prior art where a spectrophotometer associated with a probe has to be used. It is impossible to immerse the spectrophotometer and the probe in the wine for safety reasons and also due to the fact that the heat given off by the spectrophotometer will impair the wine. The recipient is advantageously a glass so as to facilitate everyday use of the probe in various different places and for example at home or at a friend's house without requiring prior preparation.

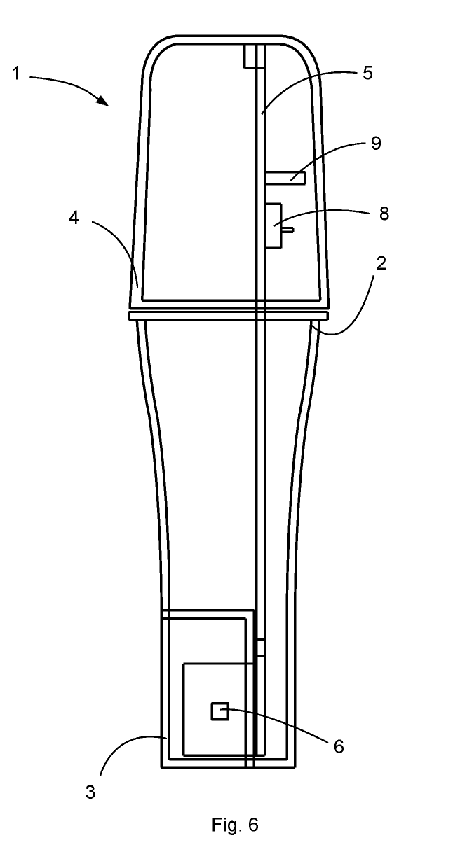

As illustrated in FIG. 6, the control device 8 is integral to the body and the measuring means in order to form a one-piece probe that is also easily transportable.

Whereas in the prior art the solutions proposed use diffraction, the inventors propose using a few specific wavelengths in order to quantify the absorption of the wine and to thus be able to compare it with other wines.

As indicated in the foregoing, the probe enables the wine to be quantified without having to impair it by a prior dilution step. The volume of analysed wine can then be tasted by the user.

The inventors also observed that characterisation of the wine from an oenological point of view can be partially approached by colorimetric parameters calculated in a colorimetric framework. It is then advantageous to measure the absorbance of the wine in three different wavelengths and to transform this data into parameters of a colorimetric framework. By means of these parameters, it is then possible to calculate a parameter representative of the body of the wine, a parameter representative of the maturity of the wine, a parameter representative of the acidity of the wine and/or a parameter representative of the tannin content.

The probe emits a plurality of light radiations in the visible range, for example in three wavelengths of the visible range. In advantageous manner, the probe is configured to emit three radiations in three different wavelengths in the visible range or more than three radiations in more than three different wavelengths.

In a particular embodiment, it is advantageous to make three measurements at three different wavelengths, for example at the 420 nm, 520 nm and 700 nm wavelengths. Other wavelengths more or less close to those proposed are natirally able to be used.

Absorbance measurements of a wine sample are made at each of these wavelengths.

Calculation of the colour density is obtained in the following manner:

D=(A₅₂₀−A₇₀₀)+(A₄₂₀−A₇₀₀) with

A₄₂₀ the absorbance measured at 420 nm.

A₅₂₀ the absorbance measured at 520 nm.

A₇₀₀ the absorbance measured at 700 nm.

Calculation of the hue is obtained in the following manner:

t=0 if max=min

t=(60°*(A₅₂₀−A₄₂₀)/(max−min)+360°) if max=A₇₀₀

t=(60° *(A₄₂₀−A₇₀₀)/(max−min)+120°) if max=A₅₂₀

t=(60°*(A₇₀₀−A₅₂₀)/(max−min)+240°) if max=A₄₂₀

max represents the maximum absorbance value between A₄₂₀, A₅₂₀ and A₇₀₀.

min represents the minimum absorbance value between A₄₂₀, A₅₂₀ and A₇₀₀.

As an alternative, determination of the colorimetric parameters representative of the wine can be performed in the CIE l*a*b colour space also called CIE LAB. The parameter a* represents the difference between red and green and the parameter b* represents the difference between blue and yellow. The parameter L* represents the clarity of the signal. Conversion into a CIE LAB framework requires the use of a set of measurements representative of a triplet of Blue, Green and Red type.

The inventors observed that a correlation exists between the maturity of the wine and the hue value, i.e. the parameter H. It is therefore advantageous to calculate a parameter representative of the maturity from the calculated hue value H. It is particularly advantageous to provide a relation that links the parameter representative of the maturity with the hue H. The relation can be chosen such that a monotone change of hue results in a monotone change of the increasing or decreasing maturity.

The inventors also observed that a correlation exists between the value of parameter a* and the acidity of the wine. The inventors propose to calculate the parameter representative of the acidity by means of parameter a*. It is particularly advantageous to provide a relation that links the parameter representative of the acidity with the parameter a*. The relation can be chosen such that a monotone change of parameter a* results in a monotone change of the increasing or decreasing maturity. The inventors also advantageously propose to weight the value of parameter a* with the calculated hue H to calculate the value of the parameter representative of the acidity. For example, the parameter a*/H can be used to calculate the value of the parameter representative of the acidity. Other weighting methods are also possible. This weighting makes it possible to take better account of the ageing of a wine, as wine tends to change colour when ageing without having an equivalent effect on the acidity.

It is also possible to provide for weighting by hue not to be performed directly by the value of H but by a compensation parameter calculated from the value of H. As the ageing of wine is not uniform, it is possible to calculate a compensation coefficient in the form C=−0.00034*H²+0.38*H−0.046. Other equations are also possible.

The inventors also observed that a correlation exists between the parameter L*, i.e. the lightness of the wine, and the body of the wine. The inventors propose to calculate the parameter representative of the body of the wine by means of the parameter L*. The inventors also propose, in advantageous manner, to weight the value of the parameter L* with the calculated hue H to calculate the value of the parameter representative of the body. For example, the parameter L*/H can be used to calculate the value of the parameter representative of the body. Other weighting methods are also possible. This weighting makes it possible to take better account of the ageing of a wine, as wine tends to change colour when ageing without having an equivalent effect on the body.

The inventors also observed that a correlation exists between the value of the parameter b* and the astringency of the wine. The inventors propose to calculate a parameter representative of the astringency of the wine, i.e. the tannin content of the wine. The inventors also propose, in advantageous manner, to weight the value of the parameter b* with the calculated hue H to calculate the value of the parameter representative of the tannin content. For example, the parameter b*/H can be used to calculate the value of the parameter representative of the body. Other weighting methods are also possible. This weighting makes it possible to take better account of the ageing of a wine, as wine tends to change colour when ageing without having an equivalent effect on its astringency.

For the parameters representative of the acidity, the body or the tannin content, it is advantageous to use a relation which is a monotone function of the associated colorimetric parameter when the hue value is constant between the different wines.

1-11. (canceled)

12. Wine probe comprising:
a sealed body configured to be immersed in wine and defining a through cavity of the sealed body,
a cover closing the sealed body,
at least one light source configured to consecutively emit at least two radiations in at least two different wavelengths, the at least one light source being positioned on a first side wall of the through cavity defined in the sealed body,
at least one light sensor positioned on a second side wall of the through cavity opposite the first side wall, the at least one light sensor being placed facing the at least one light source, the at least one light sensor being configured to measure at least two light intensities from the at least two radiations emitted by the at least one light source which have passed through the through cavity,
a control device configured to initiate emission of the at least two radiations and to collect the light intensity measurements made by the at least one light sensor, the control device being configured to transmit data relative to an analysed wine to a readout from the at least two light intensity measurements, the control device being housed in an inner volume defined by the sealed body and the cover.

13. Wine probe according to claim 12, wherein the through cavity is L-shaped in a cutting plane orthogonal to a longitudinal axis of the sealed body, the longitudinal axis connecting the through cavity and the cover mounted removable.

14. Wine probe according to claim 12, wherein the through cavity has a width comprised between 1 and 10 mm.

15. Wine probe according to claim 14, wherein the through cavity has a width comprised between 1 and 5 mm, and ideally equal to 2 mm

16. Wine probe according to claim 12, wherein:
the at least one light source is configured to successively emit the at least two radiations, with a frequency of repetition of said at least two radiations of more than 30 Hz,
the at least one light sensor is configured to perform the at least two light intensity measurements, with a frequency of repetition of more than 30 Hz,
the control device collects the at least two light intensity measurements, with a frequency of repetition of more than 30 Hz.

17. Wine probe according to claim 16, wherein the control device is configured to collect several series of at least two light intensity measurements and to calculate a statistical mean of each of the measured light intensities.

18. Wine probe according to claim 12, wherein the at least one light source comprises at least two distinct monochromatic sources.

19. Wine probe according to claim 12, wherein the at least one light source is configured to consecutively emit at least three radiations in at least three different wavelengths, and wherein the control device transforms the different measured light intensities into coordinates in a Red, Green and Blue framework.

20. Wine probe according to claim 12, wherein the at least one light source is configured to consecutively emit at least three radiations in at least three different wavelengths, and wherein the control device transforms the different measured light intensities into coordinates in a Hue, Saturation, Lightness and/or a CIELAB colorimetric framework.

21. Wine probe according to claim 12, wherein the at least one light source is configured to consecutively emit at least three radiations in at least three different wavelengths, and wherein the control device transforms the different measured light intensities into coordinates in a framework representing an acidity value, a body value and maturity value of an analysed wine and wherein these coordinates are displayed on the readout.

22. Method for measuring data relative to a wine comprising the following steps:
providing a wine probe according to claim 12,
immersing the through cavity of the wine probe in the wine so that the light emitted by the light source passes through the wine until it reaches the at least one light sensor,
emitting at least two radiations in at least two different wavelengths and measuring the at least two light intensities received by the at least one light sensor for the at least two radiations,
collecting the at least two measurements measured by the at least one light sensor,
transmitting data relative to the analysed wine to a readout from the at least two light intensity measurements.

23. Method according to claim 22, wherein:
the at least one light source is configured to consecutively emit at least three radiations in at least three different wavelengths,
the at least one light sensor is configured to measure at least three light intensity measurements from the at least three radiations.
the data relative to the analysed wine are acidity, body and maturity, this data being calculated from the at least three light intensity measurements.
